(19) United States
(12) Patent Application Publication
(10) Pub. No.: US 2007/0098788 A1
(43) Pub. Date: May 3, 2007

(54) NON-BENZODIAZEPINE HYPNOTIC COMPOSITIONS

(76) Inventors: Subhash Pandurang Gore, Sholapur 413107, Maharashtra (IN); Sudeep Kumar Agrawal, Rajpur 492010, Chhattisgarh (IN); Abhijeet Upadhye, Aurangabad 431001, Maharashtra (IN); Srinivasulu Rangineni, Mahabubnagar 509102, Andhra Pradesh (IN); Sivakumar Reddy Bommareddy, Vijayawada 520010, Andhra Pradesh (IN); Srinivas Reddy Gade, Nalgonda 508219, Andhra Pradesh (IN); Basappa Amminbavi Nagaraj, Dharwad 581201, Karnataka (IN); Gautam Bakshi, Panchkula 134112, Haryana (IN); Surjit Das, Salt Lake, Kolkata (IN); Satish Kumar Jain, Bilaspur 495001, Chhattisgarh (IN); Surya Narayana Devarakonda, Hyderabad 500047, Andhra Pradesh (IN); Bharath Reddy Deevireddy, Karnool 518511, Andhra Pradesh (IN); Eswaraiah Sajja, Hyderabad 500072, Andhra Pradesh (IN); Venkata Naga Kali Vara Prasada Raju Veturi, Hyderabad 500072, Andhra Pradesh (IN); Sreenadhacharyulu Kandala, Hyderabad 500072, Andhra Pradesh (IN); Gurvinder Singh, New Delhi 110058, Delhi (IN); Indu Bhushan, Hyderabad 500072, Andhra Pradesh (IN); Mailatur Sivaraman Mohan, Hyderabad 500072, Andhra Pradesh (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/553,342

(22) Filed: Oct. 26, 2006

Related U.S. Application Data
(60) Provisional application No. 60/745,877, filed on Apr. 28, 2006.
(60) Provisional application No. 60/746,392, filed on May 4, 2006.
(60) Provisional application No. 60/747,828, filed on May 22, 2006.
(60) Provisional application No. 60/804,949, filed on Jun. 16, 2006.
(60) Provisional application No. 60/805,019, filed on Jun. 16, 2006.
(60) Provisional application No. 60/806,266, filed on Jun. 30, 2006.

(30) Foreign Application Priority Data
Oct. 28, 2005 (IN) 1567/CHE/2005
Dec. 9, 2005 (IN) 1807/CHE/2005
Dec. 15, 2005 (IN) 1841/CHE/2005
Feb. 6, 2006 (IN) 181/CHE/2006
Feb. 20, 2006 (IN) 269/CHE/2006
Feb. 24, 2006 (IN) 308/CHE/2006

(51) Int. Cl. A61K 31/519; A61K 9/20

(52) U.S. Cl. 424/464; 514/259.3

(57) ABSTRACT

Pharmaceutical compositions comprising non-benzodiazepine hypnotic drugs or their pharmaceutically acceptable salts, solvates, enantiomers or mixtures and processes for preparing the same. The invention also includes immediate release and extended release pharmaceutical compositions comprising non-benzodiazepine hypnotic drugs, useful for sleep induction and sleep maintenance.

### INTRODUCTION TO THE INVENTION

The present invention relates to pharmaceutical compositions comprising non-benzodiazepine hypnotic drugs or their pharmaceutically acceptable salts, solvates, enantiomers or mixtures, and processes for preparing the same.

More particularly, the present invention relates to immediate release and extended release pharmaceutical compositions comprising non-benzodiazepine hypnotic drugs for sleep induction and sleep maintenance.

Non-benzodiazepine hypnotic drugs are short acting hypnotics used in the treatment of insomnia. They have hypnotic efficacy similar to that of benzodiazepines and cause less disruption of the normal sleep architecture than benzodiazepines. Psychomotor and memory impairment, respiratory depression, rebound insomnia and withdrawal symptoms upon discontinuation of non-benzodiazepines are less, compared to the longer-acting benzodiazepines. Moreover non-benzodiazepine hypnotics have a low abuse potential. In the context of the present invention, non-benzodiazepine hypnotics comprise drugs such as zolpidem, zaleplon, eszopiclone, indiplon, or pharmaceutical salts and the like, or combinations thereof.

Zolpidem is chemically N,N, 6-trimethyl-2-p-tolyl-imidazo[1,2-a]pyridine-3-acetamide, or N,N, 6-trimethyl-2-(4-methylphenyl) imidazo[1,2-a]pyridine-3-acetamide, and has structural Formula I.

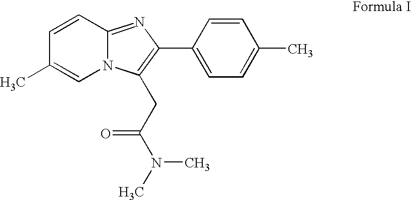

Zolpidem is a specific agonist of the ω-1 benzodiazepine (BZD) receptor. It is a non-benzodiazepine hypnotic of the imidazopyridine class having a short duration of action. It is used in the treatment of insomnia. It has a rapid onset of action (usually within 15 minutes) and has a short elimination half-life (2-3 hours). Zolpidem, in the form of its tartrate salt, is commercially available under the trade name AMBIEN® as tablets containing 5 mg and 10 mg of zolpidem base equivalent for oral administration, manufactured by Sanofi-Aventis. AMBIEN® tablets are characterized by quick and rapid release of the zolpidem. The recommended dose for adults is 10 mg immediately before bedtime. It is also available in an extended release tablet under the trade name AMBIEN CR™ 6.5 mg being recommended for elderly and 12.5 mg recommended for adults. AMBIEN CR™ is in the form of coated bilayer tablets and is indicated for sleep induction and sleep maintenance.

Zaleplon is chemically named N-[3-(3-cyanopyrazolo[1,5-α]pyrimidin-7-yl)phenyl]-N-ethylacetamide and has structural Formula II.

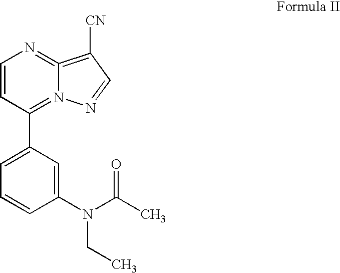

Zaleplon is a specific agonist of the ω-1 benzodiazepine (BZD) receptor. It is a non-benzodiazepine hypnotic of the pyrazolopyrimidine class and has a short duration of action. It is used in the treatment of insomnia. Zaleplon is commercially available as 5 mg and 10 mg capsules under the trade name SONATA™ and manufactured by Wyeth.

Indiplon has the chemical name N-methyl-N-(3-[3-(2-thienylcarbonyl)-pyrazolo [1,5-a]pyrimidin-7-yl] phenyl) acetamide and has structural Formula III. It is GABA-A benzodiazepine receptor agonist. It is at a pre-registration stage and will be used in the treatment of insomnia.

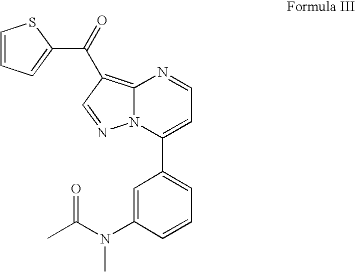

Eszopiclone is a pyrrolopyrazine derivative of the cyclopyrrolone class of non-benzodiazepine hypnotics. The chemical name of eszopiclone is (+)-(5S)-6-(chloropyridin-2-yl)-7-oxo-6,7-dihydro-5H-pyrrolo[3,4-b]pyrazin-5-yl 4-methyl-piperazine-1-carboxylate and the structural formula is shown as Formula IV. It is commercially available in the market as 1, 2 and 3 mg tablets under the brand name LUNESTA™.

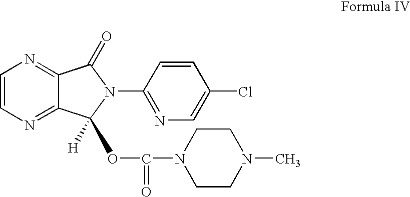

U.S. Pat. Nos. 6,638,535 and 6,514,531, and U.S. Patent Application Publication No. 2004/0258750, disclose controlled release compositions of hypnotic agents.

U.S. Pat. No. 6,242,460 describes various crystalline forms of zolpidem salts. U.S. Patent Application Publication No. 2004/0220213 describes various crystalline forms of zolpidem tartrate and processes for their preparation.

U.S. Patent Application Publication No. 2005/0119281 discloses a powder composition comprising micronized zaleplon.

U.S. Pat. No. 4,626,538 discloses generically and specifically zaleplon, a pharmaceutical composition and preparation thereof, and the use in the treatment of ameliorating anxiety.

U.S. Patent Application Publication Nos. 2005/0032818 and 2002/0072527 disclose crystalline Forms I, II, III, IV, and V of zaleplon, processes for making, pharmaceutical compositions and their use in the treatment of insomnia.

Zaleplon presents certain challenges for formulation in a rapid-onset dosage form since it has a very low solubility in aqueous media (being practically insoluble) and therefore is not readily dissolved in the gastrointestinal tract for rapid absorption when administered orally.

The amorphous forms of a number of drugs exhibit enhanced dissolution characteristics, resulting frequently in an enhanced bioavailability as compared to their crystalline counterparts. Hence, it has been the endeavor of pharmaceutical scientists to provide amorphous forms of crystalline drug substances, more specifically, thermodynamically stable forms of drug substances, which would have the strengths of the crystalline forms, viz. thermodynamic stability, and those of the amorphous form, viz. enhanced solubility, rapid onset of action and an enhanced bioavailability.

Thus, stable pharmaceutical compositions an amorphous form of non-benzodiazepine hypnotics either alone or in combination with a pharmaceutically acceptable carrier would provide a significant improvement in the treatment of insomnia and other disorders.

The pharmaceutical compositions of the present invention provide for stable immediate release and extended release pharmaceutical compositions comprising non-benzodiazepine hypnotics with desired in vitro release and in vivo absorption profiles.

### SUMMARY OF THE INVENTION

The present invention relates to pharmaceutical compositions comprising non-benzodiazepine hypnotic drugs or their pharmaceutically acceptable salts, solvates, enantiomers or mixtures and processes for preparing the same.

An aspect of the present invention provides for stable pharmaceutical compositions comprising amorphous non-benzodiazepine hypnotic drugs.

Another aspect of the present invention provides for immediate release pharmaceutical compositions comprising amorphous non-benzodiazepine hypnotic drugs.

In an embodiment, the amorphous non-benzodiazepine hypnotic drug in immediate release pharmaceutical compositions comprises amorphous zolpidem or zaleplon or eszopiclone or a pharmaceutically acceptable salt thereof.

In another embodiment, an amorphous non-benzodiazepine hypnotic in extended release pharmaceutical compositions comprises amorphous zolpidem or zaleplon or eszopiclone or a pharmaceutically acceptable salt thereof.

Still further aspect of the present invention provides for monophasic extended release compositions comprising an amorphous non-benzodiazepine hypnotic agent and a release-controlling agent.

In one embodiment, the pharmaceutical compositions of present invention comprise a solid dispersion of amorphous non-benzodiazepine hypnotic drug.

In another embodiment the present invention includes the process of preparation of amorphous forms of the non-benzodiazepine hypnotic drugs such as zolpidem or zaleplon or eszopiclone or their pharmaceutically acceptable salts.

In another embodiment the present invention includes the process of preparation of pharmaceutical compositions comprising an amorphous form of the non-benzodiazepine hypnotic drugs such as zolpidem or zaleplon or eszopiclone or their pharmaceutically acceptable salts.

In an embodiment the present invention includes the method of using the pharmaceutical compositions.

An aspect of the invention includes a composition comprising an intimate dispersion of an amorphous non-benzodiazepine hypnotic drug or a salt thereof and a polymer.

Another aspect of the invention includes a composition comprising an intimate dispersion of amorphous zaleplon and a polymer.

A further aspect of the invention includes a composition comprising an intimate dispersion of amorphous zolpidem or a salt thereof, and a polymer.

A still further aspect of the invention includes amorphous eszopiclone.

### BRIEF DESCRIPTION OF THE DRAWINGS

FIG. 1, curve A is an X-ray powder diffraction (“XRD”) pattern of crystalline zaleplon.

FIG. 1, curve B is an XRD pattern of a placebo formulation prepared according to Example 12, but omitting the zaleplon.

FIG. 1, curve C is an XRD pattern of the formulation of Example 12.

FIG. 1, curve D is an XRD pattern of Example 12 stability sample stored at 40° C. and 75% relative humidity (“RH”) for one month.

FIG. 1, curve E is an XRD pattern of Example 12 stability samples stored at 40° C. and 75% RH for two months.

FIG. 1, curve F is an XRD pattern of Example 12 stability samples stored at 40° C. and 75% RH for three months.

FIG. 2 is an XRD pattern of a placebo formulation prepared according to Example 13, but omitting the zaleplon.

FIG. 3 is an XRD pattern of the formulation of Example 13.

FIG. 4A is an XRD pattern of a placebo formulation prepared according to Example 11 but omitting the zolpidem.

FIG. 4B is an XRD pattern of the formulation of Example 11.

FIG. 4C is an XRD pattern of Example 11 formulation stability samples stored at 40° C. and 75% RH for three months.

FIG. 5 is an XRD pattern of the amorphous eszopiclone prepared in Example 4.

FIG. 6 is an XRD pattern of the eszopiclone composition prepared in Example 7.

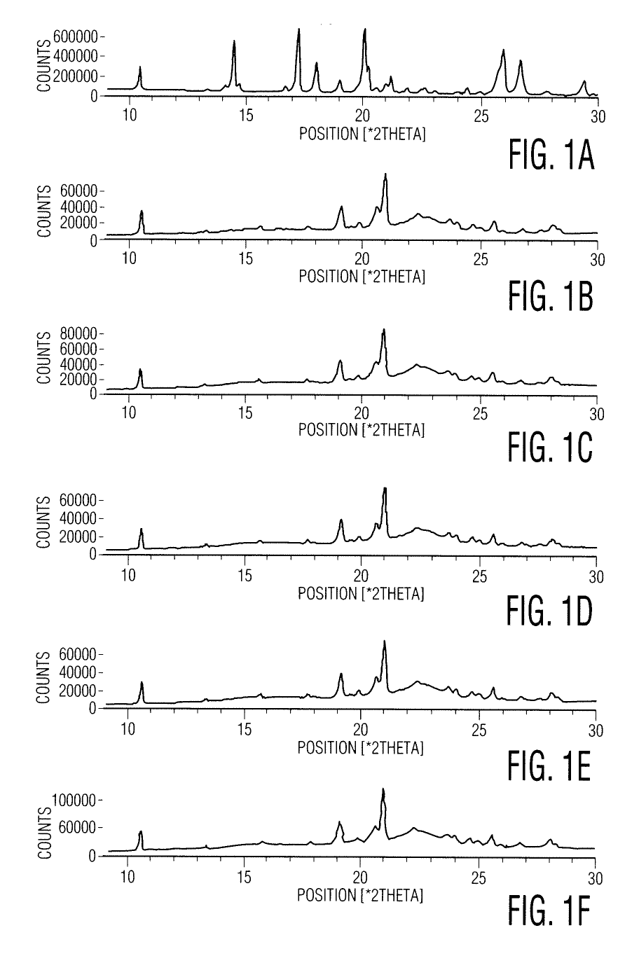

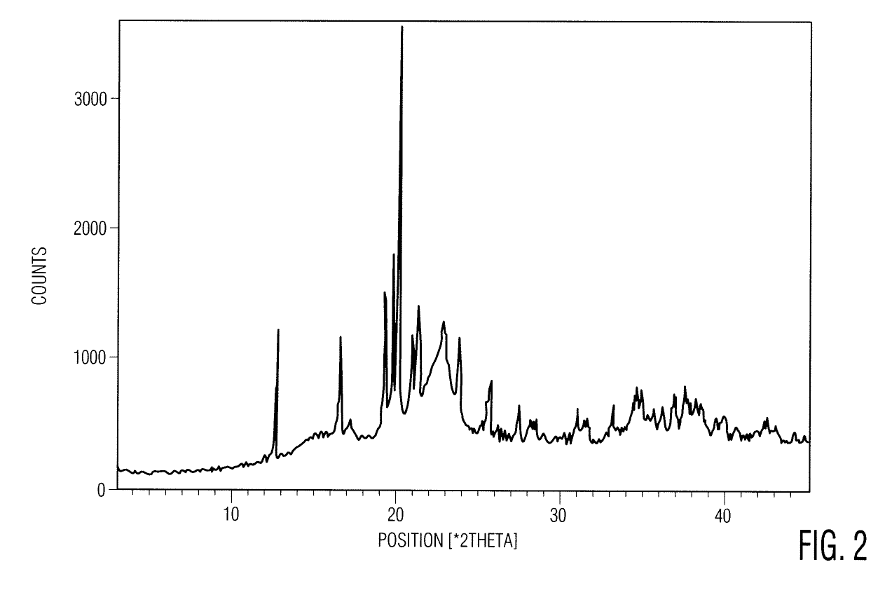

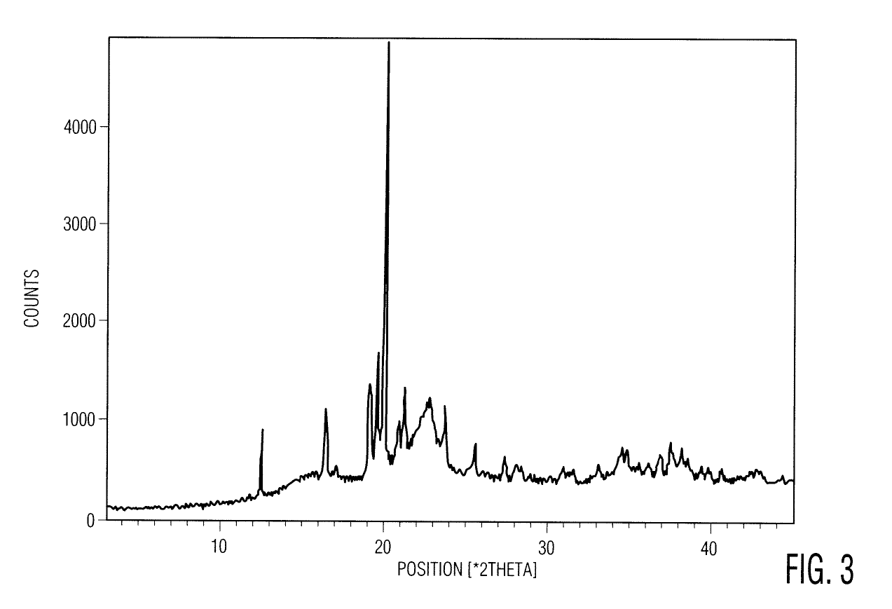

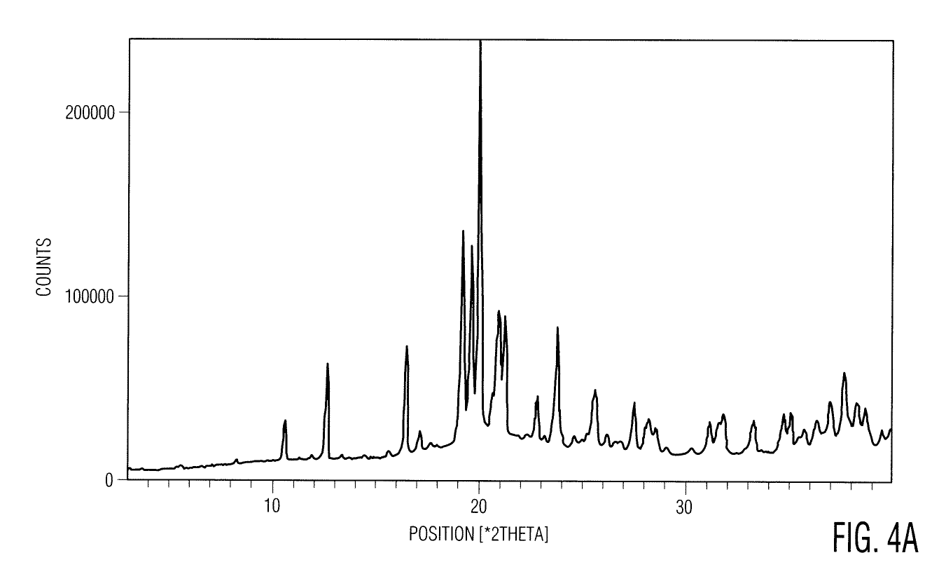

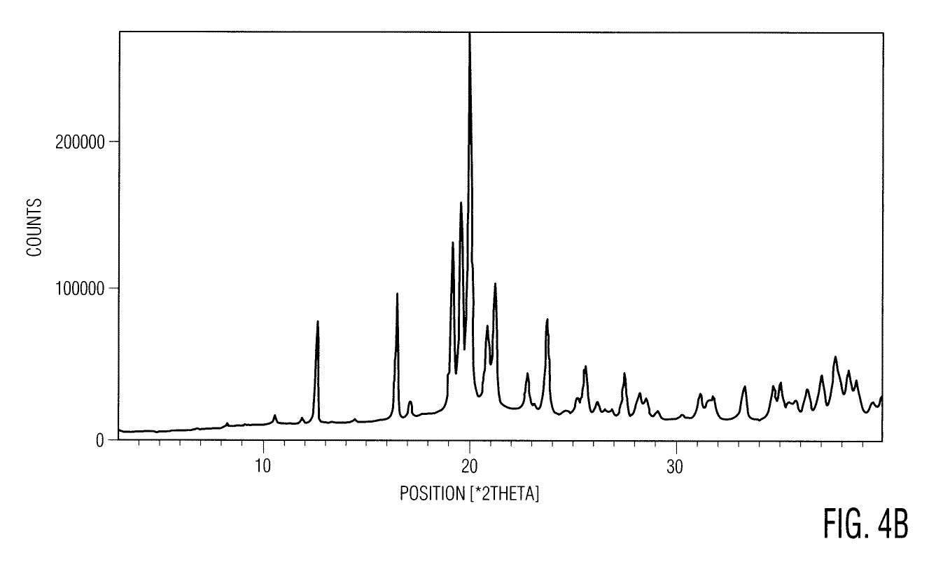

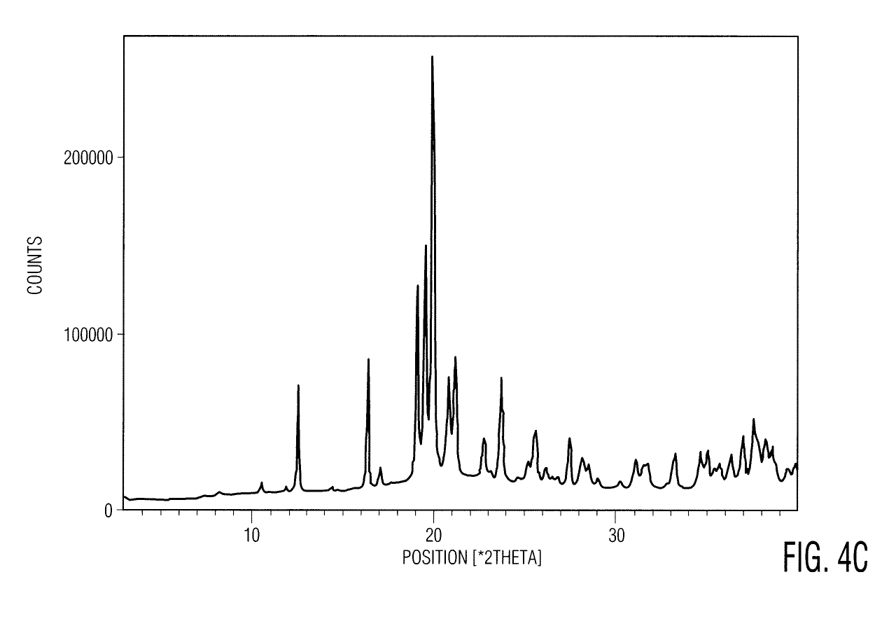

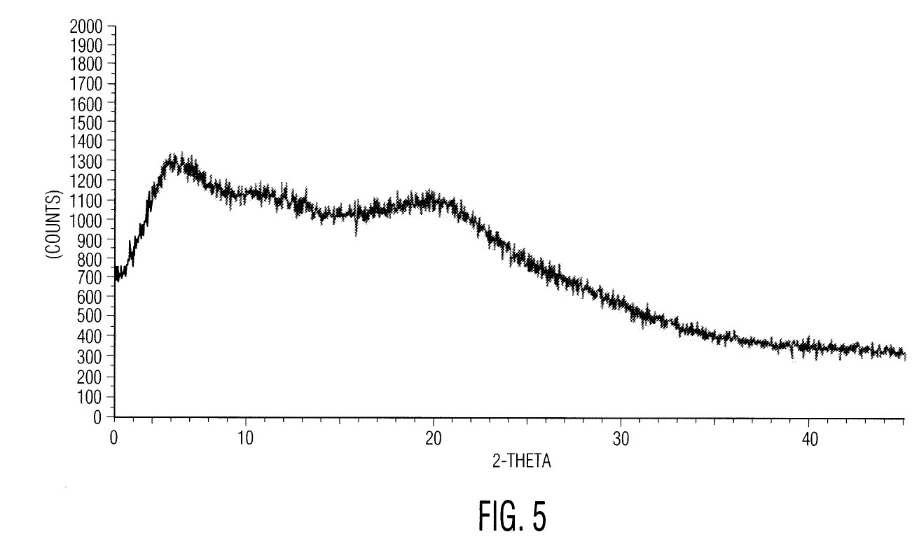

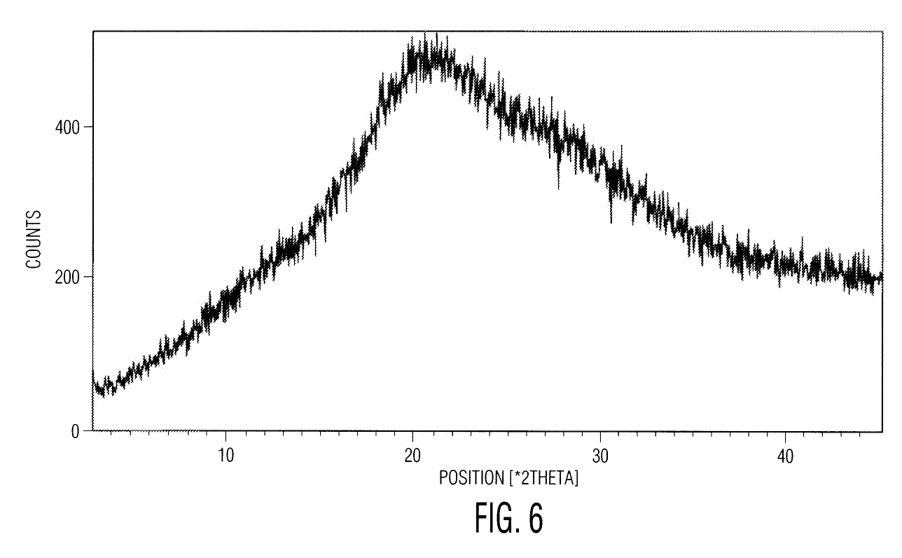

### DETAILED DESCRIPTION OF THE INVENTION

In the context of the present invention, the terms “active” or “active agent” or “active substance” or “pharmacologically active agent” or “drug” or “drug substance” may be used synonymously for an active pharmaceutical ingredient (“API”).

The present invention relates to pharmaceutical compositions comprising non-benzodiazepine hypnotic drugs or their pharmaceutically acceptable salts, solvates, enantiomers or mixtures and processes for preparing the same

An aspect of the present invention provides for stable pharmaceutical compositions comprising amorphous non-benzodiazepine hypnotic drugs.

Another aspect of the present invention provides for immediate release pharmaceutical compositions comprising amorphous non-benzodiazepine hypnotic drugs.

In an embodiment, the amorphous non-benzodiazepine hypnotic drug in immediate release pharmaceutical compositions comprises amorphous zolpidem or zaleplon or eszopiclone or its pharmaceutically acceptable salts.

In another embodiment, an amorphous non-benzodiazepine hypnotic in extended release pharmaceutical compositions comprises amorphous zolpidem or zaleplon or eszopiclone or its pharmaceutically acceptable salt.

A still further aspect of the present invention provides for monophasic extended release compositions comprising an amorphous non-benzodiazepine hypnotic agent and a release-controlling agent.

In one embodiment, the pharmaceutical compositions of present invention comprise a solid dispersion of a non-benzodiazepine hypnotic drug comprising amorphous drug.

In another embodiment the present invention includes the process of preparation of amorphous form of the non-benzodiazepine hypnotic drug such as zolpidem or zaleplon or eszopiclone or its pharmaceutically acceptable salts.

In another embodiment the present invention includes the process of preparation of pharmaceutical compositions comprising amorphous form of the non-benzodiazepine hypnotic drug such as zolpidem or zaleplon or eszopiclone or its pharmaceutically acceptable salts.

In an embodiment the present invention includes the method of using the pharmaceutical compositions.

In one embodiment of the present invention, salts of non-benzodiazepine hypnotic agents can be used, including but not limited to, hydrochloride, hydrobromide, maleate, fumarate, tartrate, hydrogen tartrate, mesylate, and tosylate salts, and the like. The salts may be crystalline or amorphous or mixtures thereof.

In an embodiment of the present invention, a tartrate salt of zolpidem is found to be particularly useful.

In one embodiment, pharmaceutical compositions of present invention comprise non-benzodiazepine hypnotic drugs in crystalline or amorphous form, or mixtures thereof.

In yet another embodiment, the starting materials for making pharmaceutical compositions comprising amorphous non-benzodiazepine hypnotic drugs can either be crystalline, amorphous, or mixtures thereof.

The pharmaceutical compositions comprising amorphous non-benzodiazepine hypnotic can be further formulated as immediate release or as extended release formulations. The pharmaceutical compositions comprising amorphous non-benzodiazepine hypnotics, with or without other pharmaceutically acceptable excipients, may be in the form powders, pellets, multi-unit particulate systems, tablets, or capsules, which are optionally coated.

In the context of the present invention, the techniques for making compositions comprising amorphous non-benzodiazepine hypnotics include, but are not limited to, melt precipitation, melt-quenching, milling, resinate formation, solid solutions and solid dispersions prepared by oven drying, tray drying, rotational drying (such as with the Buchi Rotavapor), freeze-drying, fluidized bed drying, flash drying, spin flash drying, agitated thin film drying (ATFD) and the like. Solid dispersions comprising non-benzodiazepine hypnotics prepared by rotational drying and fluidized bed drying are found to be particularly useful in aspects of the present invention.

In an embodiment, amorphous non-benzodiazepine hypnotics prepared by forming a resinate or a solid dispersion by various techniques are of particular interest for the present invention.

The amorphous non-benzodiazepine hypnotic drug can be prepared by forming a resinate or other solid intimate dispersion (such as a solid solution) by various techniques including, but not limited to, the process wherein a pharmaceutically acceptable polymer is dispersed or dissolved in a suitable solvent, typically with stirring. The active is added to this dispersion or solution after a specified time, typically with additional stirring. The mixture so obtained is evaporated at a desired temperature to obtain a dried residue comprising amorphous active. A solid intimate dispersion prepared by evaporating solvent from a solution containing a non-benzodiazepine hypnotic drug and a polymer is theoretically considered to be dispersion on the molecular level, or a solid solution.

In the context of the present invention, ion exchange resins that are useful include but are not limited to DUOLITE™ AP143/1083 (cholestyramine resin USP), polacrilin resin (AMBERLITE™ IRP 64) and polacrilin potassium (AMBERLITE™ IRP 88), all of which have been found to be particularly useful in the invention.

In another embodiment, the weight ratios of ion exchange resin to non-benzodiazepine hypnotic in the stable and taste masked pharmaceutical compositions can range from about 4:1 to 1:4, or about 2:1 to 1:2, respectively.

In yet another embodiment, the present invention provides taste-masked pharmaceutical compositions comprising a resinate formed from a non-benzodiazepine hypnotic and an ion exchange resin, optionally combined with pharmaceutically acceptable excipients to form a composition.

Further, non-benzodiazepine hypnotic drug compositions can be made into amorphous forms optionally with a pharmaceutically acceptable polymer. The weight ratios of non-benzodiazepine hypnotic drugs or salts thereof to pharmaceutically acceptable polymer can range from about 4:1 to 1:5, or about 2:1 to 1:2, or about 1:1. Frequently, a weight ratio of non-benzodiazepine hypnotic drugs or salts thereof to pharmaceutically acceptable polymer will be about 1:1 to about 1:5, such as about 1:5, or about 1:4, or about 1:3, or about 1:2, or about 1:1.5, or about 1:1.

Various pharmaceutically acceptable polymers that can be used optionally for the preparation of amorphous non-benzodiazepine hypnotic drug compositions can be either hydrophilic or hydrophobic. Useful hydrophilic or water-soluble polymers include, but are not limited to, celluloses such as hydroxypropyl methylcellulose, hydroxypropyl cellulose, polyvinylpyrrolidone (PVP), methacrylate-divinylbenzene copolymers such as DUOLITE™ AP143/1083 (cholestyramine resin USP), AMBERLITE™ IRP-64 or 88; polyhydric alcohols, polyoxyethylene derivatives, homopolymers or copolymers of N-vinylpyrrolidone (PVP) and the like. Useful hydrophobic or water-insoluble polymers are exemplified by, but are not limited to, celluloses such as ethyl cellulose, low substituted hydroxypropyl cellulose (L-HPC), crosslinked polyvinylpyrrolidone such as crospovidone, copolymers of the above polymers or mixtures of any two or more in various ratios as required without limitation.

Various pharmaceutically acceptable excipients that may form a part of resinate can be surfactants, diluents, disintegrants, hydrophilic and/or hydrophobic polymers, low molecular weight oligomers, natural products, surfactants and other commonly used excipients.

The present invention, in another embodiment, provides for stable extended release compositions comprising amorphous zolpidem and a release-controlling agent, optionally with other pharmaceutical excipients.

The extended release compositions comprising amorphous non-benzodiazepine hypnotics, with or without pharmaceutically acceptable excipients, can release the drug in the following ways: monophasic release, biphasic release, or triphasic release. Monophasic controlled release compositions are of particular interest in the present invention. Furthermore, the extended release dosage pharmaceutical compositions of non-benzodiazepine hypnotic can be formulated either as monolithic or as heterogeneous compositions and can be made into matrix systems or reservoir systems, or combinations of matrix and reservoir systems.

In the context of the present invention, the substances used for providing extended release can either be coated as a layer or coating onto the active or the active-comprising composition, or admixed or blended or adsorbed onto the active. The coating can be done by techniques known to one skilled in the art such as spray coating, dip coating, fluidized bed coating and the like.

Further layering can be done on pharmaceutical compositions of the present invention by powder coating or spray coating onto inert particles. The resulting materials may be optionally mixed or blended or adsorbed with pharmaceutically acceptable excipient(s) to be either encapsulated or compressed into tablets or mini-tablets.

In one embodiment, the present invention provides pH independent extended release zolpidem formulations, which release the drug as a monophasic profile.

In another embodiment, the extended release of the drug from the dosage form is expected to lessen the inter-individual variability with respect to the patients in which the pH of the gastrointestinal tract has been altered due to some disease conditions.

pH independent release controlling agents are used to prepare the monolithic or heterogeneous compositions exhibiting monophasic release. Release of active agent from compositions of the present invention can be modified by using rate controlling agents including, but not limited to: water soluble polymers of various grades such as celluloses including methylcellulose, carboxymethyl cellulose, hydroxypropyl methylcellulose (“HPMC”), cross-linked sodium carboxymethyl cellulose and cross-linked hydroxypropyl cellulose; carboxymethylamide; potassium methacrylate/divinylbenzene copolymer; polymethylmethacrylate; polyhydroxyalkyl methacrylate; cross-linked polyvinylpyrrolidone; gums such as agrose, gum arabic, gum ghatti, gum karaya, gum tragacanth; hydrophilic colloids such as alginates; other substances such as arbinoglactan, pectin, amylopectin, N-vinyl lactams, polysaccharides; and the like.

Water-insoluble polymers or combinations thereof used in various ratios as release controlling agents are exemplified by, but are not limited to: oils; waxes such as beeswax, carnauba wax, and microcrystalline wax; fatty alcohols such as cetostearyl alcohol, stearyl alcohol, cetyl alcohol, and myristyl alcohol; fatty acid esters such as glyceryl monostearate, glycerol distearate, glycerol monooleate, acetylated monoglycerides, tristearin, tripalmitin, cetyl esters wax, glyceryl palmitostearate, and glyceryl behenate; celluloses such as ethylcellulose, low substituted hydroxypropyl cellulose (L-HPC), cellulose acetates, and their derivatives, cellulose acylate, cellulose diacylate, cellulose triacylate, cellulose acetate, cellulose diacetate, cellulose triacetate, mono-, di- and tri-cellulose alkanylates, mono-, di-, and tri-cellulose arylates, and mono-, di- and tri-cellulose alkenylates; polymers including polymethacrylic acid based polymers and copolymers sold using the trademark EUDRAGIT®; zein; aliphatic polyesters; copolymers of the above polymers; or mixtures of any two or more in various ratios and proportions as required without limitation.

Of course, any other polymer whether hydrophilic or hydrophobic, used alone or in combination, which aids in extended release of non-benzodiazepine hypnotics, is also acceptable in the practice of this invention.

In an embodiment, the release controlling agents comprise a water-soluble polymer such as HPMC in combination with a water-insoluble polymer such as ethylcellulose. For coating the formulation according to the present invention, weight ratios of ethylcellulose to HPMC can vary from 100:0, or 75:25, or 80:20, or 70:30, or 75:25, or 65:35.

In another embodiment, the weight of the release-controlling agent ranges from about 3% w/w to about 60% w/w, or about 5% w/w to about 45% w/w, of the final composition.

The present invention, in an embodiment, provides for stable compositions comprising amorphous zaleplon, optionally with other pharmaceutical excipients.

In one embodiment, the present invention provides for pharmaceutical compositions comprising non-benzodiazepine hypnotics with defined particle sizes which aid in processibility of formulations using these compositions and result in desired dissolution and bioavailability.

A particle size distribution of D₅₀ as used herein is defined as the distribution where 50 volume percent of the particles are smaller than that size given. A particle size distribution of D₁₀ as used herein is defined as the distribution where 10 volume percent of the particles are smaller than that size given. A particle size distribution of D₉₀ as used herein is defined as the distribution where 90 volume percent of the particles are smaller than that size given. The D₅₀ value is considered to be a “mean particle size.”

“Carr index” as used herein is defined as the % compressibility, which is % ratio of difference between tapped bulk density and initial bulk density to tapped bulk density. Carr index values between about 5-15% represent materials with excellent flowability, values between about 18-21% represent fair passable flowability and values above about 40% represent poor flowability.

“Hausner ratio” as used herein is defined as the ratio of tapped density to bulk density. A Hausner ratio of less than about 1.2 indicates good flow while ratios less than about 1.5 indicate poor flow.

In an embodiment, physicochemical properties of the pharmaceutical compositions comprising non-benzodiazepine hypnotics of a defined particle size are bulk density, Carr index, Hausner ratio and the like.

In one embodiment, pharmaceutical compositions comprising non-benzodiazepine hypnotics, optionally with pharmaceutically acceptable excipients have a defined particle size wherein non-benzodiazepine hypnotic particles have D₉₀ of not more than about 250 μm.

In one embodiment, pharmaceutical compositions comprising non-benzodiazepine hypnotics, optionally with pharmaceutically acceptable excipients have a defined particle size wherein plurality of non-benzodiazepine hypnotic particles have a mean particle size (D₅₀) of about 10 μm to about 50 μm.

In yet another embodiment pharmaceutical compositions comprising non-benzodiazepine hypnotics have apparent bulk densities from about 0.5 g/ml to about 1 g/ml and tapped densities from about 0.5 g/ml to about 1.5 g/ml when tested using the methods of United States Pharmacopeia 29, United States Pharmacepeial Convention, Inc., Rockville, Md., 2005, pages 2638-2639.

Further, pharmaceutical compositions comprising non-benzodiazepine hypnotics have Carr index values from about 15% to about 20% and Hausner ratio values from about 1 to about 3.

In another embodiment, the present invention relates to pharmaceutical compositions comprising amorphous forms of non-benzodiazepine hypnotics optionally with pharmaceutically acceptable excipients.

The present invention also relates to pharmaceutical compositions comprising non-benzodiazepine hypnotics, with or without pharmaceutically acceptable excipients, forming compositions where the drug is in an amorphous form as shown by X-ray powder diffraction studies. X-ray powder diffraction (“XRD”) patterns described herein were obtained using a PAN analytical X-Ray Diffractometer (Model: X'Pert PRO TM) and X'Celerator detector using copper k-alpha radiation (1.541 Å wavelength).

The pharmaceutical compositions of the present invention may further contain one or more diluents to makeup the final composition mass so that it becomes easier for the patient and the caregiver to handle.

Common diluents that can be used in pharmaceutical formulations comprise microcrystalline cellulose (“MCC”), silicified MCC (e.g. Prosolv™ HD 90), microfine cellulose, lactose, starch, pregelatinized starch, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, and the like.

The pharmaceutical compositions may further include a disintegrant. Disintegrants include but are not limited to methyl cellulose, microcrystalline cellulose, carboxymethyl cellulose calcium, carboxymethyl cellulose sodium (e.g. Ac-Di-Sol®, Primellose®), crospovidone (e.g. Kollidon®, Polyplasdone®), povidone (e.g., the K-30 grade), guar gum, magnesium aluminum silicate, colloidal silicon dioxide (Aerosil®), polacrilin potassium, starch, pregelatinized starch, sodium starch glycolate (e.g. Explotab®) and sodium alginate.

The compositions may further include but are not limited to pharmaceutically acceptable glidants, lubricants, opacifiers, colorants, flavors and other commonly used excipients.

Non-limiting examples of suitable solvents that can be used for preparing resinate, granulation, layering, coating, and various amorphization techniques in the present invention include water, methanol, ethanol, isopropyl alcohol, acetone, propanol, butanol, dichloromethane, ethyl acetate, butyl acetate, propyl acetate, and the like or mixtures thereof.

The following examples will further illustrate certain specific aspects and embodiments of the invention in greater detail and are not intended to limit the scope of the invention.

### EXAMPLE 1

### Preparation of an Amorphous Zolpidem Tartrate Composition

| Ingredients | Quantity/Batch (g) |
|---|---|
| Zolpidem tartrate | 2 |
| Polyvinylpyrrolidone (Povidone K- | 6 |
| 30) | |
| Isopropyl alcohol | 160 |

Manufacturing process:
1. PVP K-30 was dispersed in isopropyl alcohol with stirring.
2. To the dispersion of step 1, zolpidem tartrate was added with stirring.
3. The dispersion of step 2 was evaporated in a rotary vacuum evaporator at a temperature of 50-52° C. until loss on drying at 105° C. was not more than 3% w/w.
4. The residue was passed through an ASTM #40 mesh sieve.

### EXAMPLE 2

### Preparation of a Zaleplon Amorphous Composition by dry Distillation

5 g of zaleplon, 5 g of povidone (PVP K-30) and 40 ml of dichloromethane were charged into a round bottom flask and stirred for 15-30 minutes to obtain a solution. The resultant solution was transferred into a Buchi Rotavapor and the solvent was distilled to dryness at about 35-40° C. under a reduced pressure of about 650-700 mm Hg, followed by drying the solid obtained at 30-35° C. under a reduced pressure of about 650-700 mm Hg for 45-90 minutes to afford 9.3 grams of the desired amorphous mixture.

### EXAMPLE 3

### Process for the Preparation of Eszopiclone of Desired Particle Size

50 g of eszopiclone and 500 ml of acetonitrile were charged into a round bottom flask followed by heating to about 75° C.-80° C. for about 5-15 minutes. The resultant solution was cooled to about 25-35° C. followed by stirring for about 15-45 minutes. Separated solid was filtered and washed with 50 ml of acetonitrile followed by drying at about 55-65° C. over about 1-2 hours to afford 33.2 g of eszopiclone of the desired particle size. The particle size distribution as measured by a laser light scattering instrument (Malvern Instruments Ltd., Malvern, Worcestershire, United Kingdom) was: D₁₀=25.7 μm, D₅₀=92.9 μm, D₉₀=216.1 μm and bulk density before tapping was 0.56 g/ml and after tapping was 0.72 g/ml.

Particle size, bulk density, and other data from repeating the process described above is shown below:

| | | | | Apparent | Tapped | | |
|---|---|---|---|---|---|---|---|
| Run | Particle size | | | Bulk | Bulk | Carr | Hausner |
| No. | D₁₀ | D₅₀ | D₉₀ | Density | Density | Index | Ratio |
| 1 | 25.3 | 98.1 | 206.3 | — | — | — | — |
| 2 | 11.0 | 63.8 | 139.9 | — | — | — | — |
| 3 | 17.6 | 56.8 | 103.1 | — | — | — | — |
| 4 | 24.4 | 77.8 | 157.3 | — | — | — | — |
| 5 | 25.7 | 92.9 | 216.1 | 0.557 | 0.717 | 22.3 | 1.29 |
| 6 | 2.5 | 25.3 | 69.5 | — | — | — | — |

Particle sizes are in μm and densities in g/ml units.

### EXAMPLE 4

### Process for Preparation of Amorphous Eszopiclone

1 g of eszopiclone was charged into a crucible followed by placing into a muffle furnace and heated to about 204-205° C. The resultant molten liquid was allowed to stand for about 5 minutes. The crucible containing the molten eszopiclone was taken from muffle furnace followed by pouring it onto a stainless steel plate that was at room temperature. The eszopiclone sample was then cooled to about 0° C. Solidified eszopiclone was removed from the plate to afford 1 g of the desired eszopiclone amorphous form.

### EXAMPLE 5

### Preparation of Amorphous Composition of Zolpidem Tartrate with Povidone Using Rotavapor

2.5 g of zolpidem tartrate, 2.5 g of povidone (PVP K-30), and 90 ml methanol were taken into a round bottom flask equipped with half-moon teflon blade type agitator at about 28° C. The mixture was stirred for 20 minutes to obtain a solution. The solution was changed into a Buchi Rotavapor flask followed by distillation of solvent to dryness at about 50° C. under a vacuum of about 550 mm Hg. The obtained solid was dried at about 55° C. under a vacuum of 12 mm Hg to afford 5.0 g of the title composition.

### EXAMPLE 6

### Preparation of Amorphous Composition of Zolpidem Tartrate with Povidone Using Spray Dryer

5.0 g of zolpidem tartrate, 5.0 g of povidone (PVP K-30) and 180 ml of methanol were charged into a round bottom flask equipped with a half-moon teflon blade type agitator at 28° C. The mixture was stirred for 20 minutes to get a solution. The resultant solution was subjected to spray drying and the solvent was evaporated by maintaining the feed pump at about 10 rpm, aspirator at about 1000 rpm, inlet air temperature at about 80° C., outlet air temperature at about 40° C. under a N₂ pressure of about 2 kg/cm² to afford 3.1 g of the title composition.

### EXAMPLE 7

### Amorphous Eszopiclone Composition

40 ml of dichloromethane, 3 g of polyvinylpyrrolidone (PVP K-30) and 3 g of eszopiclone were charged into a round bottom flask at 25-35° C. The mixture was stirred for about 15-30 minutes for dissolution followed by filtration of the solution through filter paper. The filtrate was distilled to dryness in a Buchi Rotavapor flask under reduced pressure of about 25-105 Torr at below 40° C. The solid obtained was dried for about 30-60 minutes in the Buchi Rotavapor under reduced pressure at below 40° C. to afford the desired amorphous composition of eszopiclone.

### EXAMPLE 8

### Preparation of Zolpidem Resinate

| Ingredients | Quantity/Batch (g) |
|---|---|
| Zolpidem tartrate | 15 |
| Polacrilin resin | 30 |
| (Amberlite ™ IRP 64)* | |
| Water | 300 |

*Amberlite ™ IRP 64 is manufactured by Rohm and Haas Co., USA

Manufacturing process:
1. Zolpidem tartrate was dispersed in water under stirring.
2. Polacrilin resin was added to dispersion of step 1 and the mixture was continuously stirred for 1-2 hours.
3. The dispersion of step 2 was filtered and the residue obtained was dried at 65±5° C. in a tray drier until a loss on drying (LOD) below 7% w/w was obtained at 105° C. using a halogen moisture balance.
4. Finally the dried residue was sifted through an ASTM #40 mesh sieve.
   Taste masking evaluation studies.
Number of subjects: 5
Bitterness score: 1-10 (1=absence of bitterness to 10=highly bitter)

It was observed that a zolpidem-resin physical mixture having the same bulk composition (average bitterness score 8) was about two times more bitter than the zolpidem resinate prepared in this example (average bitterness score 4).

### EXAMPLE 9

### Preparation of Controlled Release Zolpidem 12.5 mg Tablets Comprising Resinate

| | |
|---|---|
| | Quantity/Batch (g) |
| Ingredients | (1000 Tablets) |

Core

| | |
|---|---|
| Zolpidem resinate (Example 8) | 50.6 |
| Hydroxypropyl methylcellulose 50 cps | 37.5 |
| Colloidal silicon dioxide | 0.8 |
| Magnesium stearate | 1.5 |
| Lactose monohydrate | 59.6 |
| Average tablet weight (mg) | 150 |

Coating

| | |
|---|---|
| Eudragit L100 D55* (Acryl-Eze ™)* | 10.5 |
| Hydroxypropyl methylcellulose | 4.5 |
| Water | q.s. |

*Acryl-Eze ™ is a formulated coating composition manufactured by Colorcon.

Manufacturing process:
1. Zolpidem resinate, hydroxypropyl methylcellulose and lactose were sifted through an ASTM #40 mesh sieve.
2. Blend of step 1 was lubricated with colloidal silicon dioxide and magnesium stearate.
3. Lubricated blend of step 2 was compressed into tablets using 7.5 mm, round shaped, biconvex punches.
4. The tablets were coated using a dispersion of Eudragit and hydroxypropyl methylcellulose in water with coating parameters as follows:
   Inlet air temperature: 60±5° C.
   Bed temperature: 45±5° C.
   Pan speed: 3-7 rpm.
In vitro dissolution study conducted with Example 8 tablets:

Medium: 0.01 N HCl

Apparatus: USP Type-II (described in Test 711-Dissolution, United States Pharmacopeia 29, United States Pharmacopeial Convention, Inc., Rockville, Md., 2005)

Volume: 500 ml

Speed: 75 rpm

| Time (hours) | % Drug Released |
|---|---|
| 0 | 0 |
| 0.5 | 32 |
| 1 | 50 |
| 2 | 72 |
| 4 | 95 |

### EXAMPLE 10

### Zolpidem Tartrate 12.5 mg Extended Release Tablets Comprising Amorphous Zolpidem Tartrate

| | |
|---|---|
| | Quantity/Batch (g) |
| Ingredients | (1000 tablets) |

Core

| | |
|---|---|
| Amorphous zolpidem tartrate (Example 1) | 38.7 |
| Hydroxypropyl methylcellulose 50 cP | 62.5 |
| Colloidal silicon dioxide | 1.3 |
| Magnesium stearate | 2.5 |
| Lactose monohydrate | 145 |
| Average tablet weight (mg) | 250 |

Coating

| | |
|---|---|
| Eudragit L100 D55 (Acryl-Eze ™) | 17.5 |
| Hydroxypropyl methylcellulose | 7.5 |
| Water | q.s. |

Manufacturing process:
1. Amorphous zolpidem tartrate composition, hydroxypropyl methylcellulose and lactose were sifted through an ASTM #40 mesh sieve.
2. Blend of step 1 was lubricated with colloidal silicon dioxide and magnesium stearate.
3. Lubricated blend of step 2 was compressed into tablets using 8.65 mm, round shaped, biconvex punches.
4. The tablets were coated using a dispersion of Eudragit and hydroxypropyl methylcellulose in water with coating parameters as follows:
   Inlet air temperature: 60±5° C.
   Bed temperature: 45±5° C.
   Pan speed: 3-7 rpm.

### EXAMPLE 11

### Zolpidem Tartrate 12.5 mg Extended Release Tablets Comprising Amorphous Zolpidem Tartrate

| | |
|---|---|
| | Quantities/Batch (Kg) |
| Ingredients | (150,000 Tablets) |

Drug Solution

| | |
|---|---|
| Zolpidem tartrate | 1.9 |
| Polyvinylpyrrolidone (Povidone K-30) | 3.8 |
| Methanol | 74.8 |

Dry Mix

| | |
|---|---|
| Lactose monohydrate (Flow | 26.8 |
| Lac ® 100) | |
| Hydroxypropyl methylcellulose 50 cP | 9.9 |
| (Methocel E 50 LV) | |

Blending and Lubrication

| | |
|---|---|
| Lactose anhydrous (DCL-21) | 2.3 |
| Colloidal silicon dioxide | 0.2 |
| (Aerosil ® 200) | |
| Magnesium stearate | 0.2 |

Film Coating

| | |
|---|---|
| Opadry Blue 03B50680* | 1.4 |
| Isopropyl alcohol | 11.7 |
| Methylene chloride | 7.8 |

*Opadry is a pigmented ready-to-use coating system containing hydroxypropyl methylcellulose as a film former, manufactured by Colorcon.

Manufacturing process:
1. Zolpidem tartrate and PVP were dissolved in methanol under stirring.
2. Lactose monohydrate and HPMC were loaded into a fluid bed processor.
3. Mixture of step 2 was granulated with solution of step 1 in a fluid bed processor.
4. The granules were dried in fluid bed processor to a loss on drying below 3% w/w.
5. The dried granules were mixed with lactose anhydrous, colloidal silicon dioxide and magnesium stearate by blending for 10 minutes.
6. Blend of step 5 was compressed into tablets using 9 mm standard concave punches.
7. Tablets of step 6 were coated with Opadry dispersion in isopropyl alcohol and methylene chloride using a perforated coating pan to get a weight build-up of 2-3.5% w/w.
   In vitro dissolution test was conducted under the following conditions:
Medium: 0.01 N HCl
Apparatus: USP Apparatus II (Paddle)
Volume: 500 ml

Speed: 75 rpm

| Time (hours) | % Drug Released |
|---|---|
| 0 | 0 |
| 0.5 | 20 |
| 1 | 31 |
| 2 | 49 |
| 3 | 70 |
| 4 | 102 |

### EXAMPLE 12

### Composition of Zaleplon Capsules 10 mg

| | |
|---|---|
| | Quantity/Batch (g) |
| Ingredients | (500 Capsules) |

Solid dispersion

| | |
|---|---|
| Zaleplon | 5 |
| Polyvinylpyrrolidone (Povidone ® K-30)* | 7.5 |
| Methylene chloride | 75 |

Blending

| | |
|---|---|
| Lactose anhydrous (DCL-21)** | 11.3 |
| Microcrystalline cellulose (Avicel ® PH 112)# | 11.4 |
| Pregelatinized starch (Starch1500 ®)## | 9 |
| Sodium lauryl sulphate | 0.05 |

Lubrication

| | | |
|---|---|---|
| Colloidal silicon dioxide (Aerosil ® 200)$ | 0.5 | |
| Magnesium stearate | 0.3 | |
| Average capsule weight | 90 | mg |

*BASF manufactures polyvinylpyrrolidone (Povidone K-30).
**DCL-21 is manufactured by DMV Inc.
#FMC Biopolymer manufactures Avicel ® PH 112.
##Starch1500 ® is manufactured by Colorcon.
$Aerosil ® 200 is manufactured by Degussa.

Manufacturing process:
1. Zaleplon and povidone were dissolved in methylene chloride under stirring to form a clear solution.
2. The solution of step 1 was transferred to a flask and the solvent was removed using a Buchi Rotavapor evaporator under vacuum.
3. The solid dispersion obtained in step 2 was milled in an air jet mill.
4. The solid dispersion of step 3, microcrystalline cellulose, lactose anhydrous, pregelatinized starch, sodium lauryl sulphate and colloidal silicon dioxide were co-sifted through an ASTM #40 mesh sieve and mixed together.
5. Blend of step 4 was lubricated with magnesium stearate by mixing.
6. Lubricated blend of step 5 was filled into size “4” hard gelatin capsules with an average weight of 90 mg by an automated capsule filling machine.
   In vitro dissolution test was conducted under the following conditions:
Medium: Phosphate buffer pH 6.8
Apparatus: USP Apparatus 11-Paddle
Volume: 900 ml

Speed: 50 rpm

% Drug Released

| | Example 10 (Zaleplon | SONATA ® 10 mg |
|---|---|---|
| Time (minutes) | 10 mg Capsules) | Capsules |
| 0 | 0 | 0 |
| 10 | 76 | 83 |
| 20 | 97 | 98 |
| 30 | 100 | 98 |

### EXAMPLE 13

### Composition of Zaleplon Capsules 10 mg

| Ingredients | mg/Capsule |
|---|---|
| Zaleplon | 10 |
| Polyvinylpyrrolidone (Povidone K-30) | 5 |
| Lactose monohydrate (Flow Lac ® 100)* | 27 |
| Microcrystalline cellulose (Avicel ® PH 112) | 28.4 |
| Pregelatinized starch (Starch1500 ®) | 18 |
| Sodium lauryl sulphate | 0.1 |
| Colloidal silicon dioxide (Aerosil ® 200) | 1 |
| Magnesium stearate | 0.5 |
| Dichloromethane | q.s. |

*Flow Lac ® 100 is manufactured Meggle Pharma.

Manufacturing Process:
1. Lactose monohydrate, microcrystalline cellulose and pregelatinized starch were sifted through an ASTM #40 mesh sieve.
2. Zaleplon, PVP K 30 and sodium lauryl sulphate were dissolved in dichloromethane under stirring until a clear solution formed.
3. Blend of step 1 was granulated with solution of step 2.
4. The granulate of step of step 3 was dried at 60° C. to a loss on drying (“LOD”) less than 2% w/w, determined using a halogen moisture balance at 105° C.
5. The dried granules were sifted through an ASTM #30 mesh sieve and lubricated with colloidal silicon dioxide and magnesium stearate.
6. Lubricated blend of step 5 was filled into size “4” hard gelatin capsules by an automated capsule filling machine.
   In vitro dissolution test was conducted under the following conditions:
Medium: pH 4.5 acetate buffer
Apparatus: USP Apparatus II (Paddle)
Volume: 900 ml

Speed: 50 rpm

| Time (minutes) | % Drug Released |
|---|---|
| 0 | 0 |
| 10 | 89 |
| 15 | 98 |

### EXAMPLE 14

### Preparation of Solid Dispersion of Zaleplon

| Ingredients | Quantity (mg) |
|---|---|
| Zaleplon | 10 |
| Polyvinylpyrrolidone (Povidone K-30) | 10 |
| Lactose monohydrate (Flow Lac ® 100) | 25 |
| Microcrystalline cellulose (Avicel ® PH 112) | 25.1 |
| Pregelatinized starch (Starch 1500 ®) | 18 |
| Sodium lauryl sulphate | 0.1 |
| Dichloromethane | q.s. |

Manufacturing process:
1. Lactose monohydrate, microcrystalline cellulose, pregelatinized starch and sodium lauryl sulphate were sifted through an ASTM #40 mesh sieve.
2. Zaleplon and PVP K 30 were dissolved in dichloromethane under stirring until a clear solution formed.
3. The solution of step 2 was sprayed onto step 1 ingredients in a fluidized bed processor.
4. The mixture of step 3 was dried at 60° C. to a loss on drying (LOD) less than 2% w/w, as determined using a halogen moisture balance at 105° C.

### EXAMPLE 15

### Composition of Zaleplon Capsules 10 mg with zaleplon having D₉₀ of 195 μm

| Ingredients | mg/Capsule |
|---|---|
| Zaleplon | 10 |
| Lactose monohydrate (Flow Lac ® 100) | 30 |
| Microcrystalline cellulose (Avicel ® PH 112) | 28 |
| Pregelatinized starch (Starch1500 ®) | 18 |
| Sodium lauryl sulphate | 2 |
| Colloidal silicon dioxide (Aerosil ® 200) | 1 |
| Magnesium stearate | 1 |

Manufacturing process:
1. Zaleplon, lactose monohydrate, microcrystalline cellulose, pregelatinized starch and sodium lauryl sulphate were sifted through an ASTM #40 mesh sieve.
2. Colloidal silicon dioxide and magnesium stearate were separately sifted through an ASTM #60 mesh sieve.
3. The blend of step 1 was mixed with lubricants of step 2 in a double cone blender for 10 minutes.
4. Lubricated blend of step 4 was filled into capsules of size “4” by an automated capsule filling machine.

### EXAMPLE 16

### Composition of Amorphous Eszopiclone Tablets

The excipients 300 g of lactose, 300 g of calcium phosphate, 100 g of microcrystalline cellulose, and 20 g of croscarmellose sodium are mixed together in an octagonal blender for 5 minutes. 10 g of eszopiclone from Example 4 is mixed geometrically with 10 g of the above excipient blend in a polyethylene bag for 5 minutes, then mixed with another 20 g of above excipient blend in a polyethylene bag for 5 minutes and this blend is added to the octagonal blender and mixed with the remaining excipient blend for 5 minutes. To this blend, 10 g of colloidal silicon dioxide and 10 g of magnesium stearate are added and mixed for 5 minutes. This lubricated blend is compressed into 5 mm standard concave round tablets with an average weight of 75 mg to get eszopiclone 1 mg tablets or compressed as 8.5 mm standard concave round tablets with an average weight of 225 mg to get eszopiclone 3 mg tablets. Tablets are coated with a 10% aqueous dispersion of Opadry white (manufactured by Colorcon Asia Pvt. Limited, India) to get a weight build up of 2.5% w/w in a automated coating pan.

### EXAMPLE 17

### Composition of Amorphous Eszopiclone-pvp Tablets

The excipients 300 g of lactose, 300 g of calcium phosphate, 90 g of microcrystalline cellulose, and 20 g of croscarmellose sodium are mixed together in an octagonal blender for 5 minutes. 20 g of eszopiclone-pvp mixture from Example 7 is mixed geometrically with 20 g of the above excipient blend in a polyethylene bag for 5 minutes, then mixed with another 40 g of the above excipient blend in a polyethylene bag for 5 minutes and this blend is added to the octagonal blender and mixed with the remaining excipient blend for 5 minutes. To this blend 10 g of colloidal silicon dioxide and 10 g of magnesium stearate are added and mixed for 5 minutes. This lubricated blend is compressed into 5 mm standard concave round tablets with an average weight of 75 mg to get eszopiclone 1 mg tablets or compressed as 8.5 mm standard concave round tablets with an average weight of 225 mg to get eszopiclone 3 mg tablets. Tablets are coated with a 10% aqueous dispersion of Opadry white to get a weight build up of 2.5% w/w in an automated coating pan.

### EXAMPLE 18

The excipients 300 g of lactose, 300 g of calcium phosphate, 74 g of microcrystalline cellulose, and 20 g of croscarmellose sodium are mixed together in a rapid mixer granulator for 5 minutes. A solution of 10 g of eszopiclone from Example 3 dispersed in isopropyl alcohol along with 1 g of sodium lauryl sulphate in water is added to the dry excipient mixture and granulated to form wet granules. Wet granules are dried at a temperature of 50° C. in a fluid bed drier and dried granules are sifted through a 40 mesh ASTM sieve. Sifted granules are mixed with 25 g of microcrystalline cellulose for 3 minutes in an octagonal blender and to this blend 10 g of colloidal silicon dioxide and 10 g of magnesium stearate are added and mixed for 2 minutes. This lubricated blend is compressed as 5 mm standard concave round tablets with an average weight of 75 mg to get eszopiclone 1 mg tablets or compressed as 8.5 mm standard concave round tablets with an average weight of 225 mg to get eszopiclone 3 mg tablets. Tablets are coated with 10% aqueous dispersion of Opadry white to get a weight build up of 2.5% w/w in an automated coating pan.

We claim:

1. A composition comprising an intimate dispersion of an amorphous non-benzodiazepine hypnotic drug or a salt thereof and a polymer.

2. The composition of claim 1, wherein an intimate dispersion is prepared by removing solvent from a solution comprising a non-benzodiazepine hypnotic drug and a polymer.

3. The composition of claim 1, wherein a polymer comprises an ion exchange resin.

4. The composition of claim 1, wherein a polymer comprises a hydrophilic polymer.

5. The composition of claim 1, wherein a polymer comprises a polyvinylpyrrolidone.

6. The composition of claim 1, wherein a non-benzodiazepine hypnotic drug comprises zaleplon.

7. The composition of claim 1, wherein a non-benzodiazepine hypnotic drug comprises zolpidem or a salt thereof.

8. The composition of claim 1, wherein a weight ratio of non-benzodiazepine hypnotic drug or a salt thereof to polymer is about 4:1 to 1:5.

9. The composition of claim 1, wherein a weight ratio of non-benzodiazepine hypnotic drug or a salt thereof to polymer is about 1:1 to about 1:5.

10. The composition of claim 1, further comprising at least one pharmaceutically acceptable excipient.

11. A composition comprising an intimate dispersion of amorphous zaleplon and a polymer.

12. The composition of claim 11, wherein a polymer comprises an ion exchange resin.

13. The composition of claim 11, wherein a polymer comprises a polyvinylpyrrolidone.

14. The composition of claim 11, wherein a weight ratio of zaleplon to polymer is about 1:1 to about 1:5.

15. The composition of claim 11, wherein a polymer comprises a polyvinylpyrrolidone and a weight ratio of zaleplon to polymer is about 1:1.5

16. The composition of claim 11, wherein an intimate dispersion is prepared by removing solvent from a solution comprising zaleplon and having a polymer dissolved or dispersed therein.

17. The composition of claim 11, further comprising at least one pharmaceutically acceptable excipient.

18. A composition comprising an intimate dispersion of amorphous zolpidem or a salt thereof, and a polymer.

19. The composition of claim 18, wherein a polymer comprises an ion exchange resin.

20. The composition of claim 18, wherein a polymer comprises a polyvinylpyrrolidone.

21. The composition of claim 18, wherein a weight ratio of zolpidem or a salt thereof to polymer is about 1:1 to about 1:5.

22. The composition of claim 18, wherein a polymer comprises a polyvinylpyrrolidone and a weight ratio of zolpidem or a salt thereof to polymer is about 1:2.

23. The composition of claim 18, wherein an intimate dispersion is prepared by removing solvent from a solution comprising zolpidem or a salt thereof and having a polymer dissolved or dispersed therein.

24. The composition of claim 18, further comprising at least one pharmaceutically acceptable excipient.

25. Amorphous eszopiclone.

26. Amorphous eszopiclone of claim 25, in an intimate dispersion with a polymer.
